(19) United States
(12) Patent Application Publication
(10) Pub. No.: US 2025/0154318 A1
(43) Pub. Date: May 15, 2025

(54) PHTHALIMIDINE- CONTAINING CARDO BISPHENOLS BEARING PENDANT FURYL GROUP AND POLYMERS THEREFROM

(71) Applicant: Council of Scientific & Industrial Research, New Delhi (IN)

(72) Inventors: Deepak Mannusing Maher, Pune (IN); Aniket Anil Talanikar, Pune (IN); Samadhan Suresh Nagane, Pune (IN); Prakash Purushottam Wadgaonkar, Pune (IN)

(21) Appl. No.: 18/722,241

(22) PCT Filed: Dec. 22, 2022
(86) PCT No.: PCT/IN2022/051116

§ 371 Date: Jun. 20, 2024

(30) Foreign Application Priority Data
Dec. 25, 2021 (IN) 202111061091

(51) Int. Cl. C08G 63/685; C07D 405/06

(52) U.S. Cl. CPC C08G 63/6858; C07D 405/06

(57) ABSTRACT

The present invention relates to phthalimidine-containing bisphenol monomers bearing pendant furyl group of Formula (I) and (co) polymers of Formula (II) obtained by polycondensation of varying mixtures of monomer of Formula (I) and bisphenol-A with aromatic diacid chlorides and the processes for the preparation thereof.

### FIELD OF THE INVENTION

The present invention relates to bisphenol monomers bearing pendant furyl group and step-growth polymers therefrom and the processes for the preparation thereof. More particularly, the present invention relates to bisphenol monomers bearing pendant furyl group of Formula (I) and (co) polymers of Formula (II) obtained by polycondensation of a mixture of bisphenol of Formula (I) and bisphenol-A with aromatic diacid chlorides and process for the preparation thereof.

### BACKGROUND AND PRIOR ART OF THE INVENTION

Bisphenols are of particular interest as monomers of practical utility due to their usefulness as building blocks for the synthesis of industrially relevant polymers such as polycarbonates, polyesters, poly (arylene ether) s, cyanate esters, epoxy resins, etc. In particular, bisphenols bearing pendant (clickable) functional groups are of significant importance as they offer access to step-growth polymers fitted with respective pendant functional groups which could subsequently be exploited in post-modifications with appropriate coupling partners. Bisphenols bearing pendant clickable functional groups such as alkene, propargyloxy, norbornenyl, furyl, azido or maleimido are reported in the literature. Amongst these, bisphenols bearing pendant furyl group have unique advantages due to their utility in thermally triggered Diels-Alder (DA) click reaction and retro-Diels-alder (rDA) reaction with maleimide and triazolinedione (TAD) reagents.

US Patent, U.S. Pat. No. 10,239,865B2 discloses bisphenol monomers of Formula I with pendant maleimide group connected via alkylene spacer and preparation thereof. Also, it discloses polymers based on bisphenol monomers containing pendant clickable maleimide group. Further, it provides a process for the preparation of polymers possessing pendant clickable maleimide groups based on bisphenols containing pendant maleimide group. Formula (I) wherein, x is an integer selected from 0 to 10.

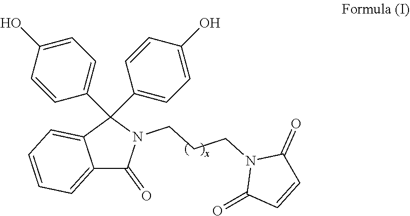

Article titled, “A new cardobisphenol monomer containing pendant azido group and the resulting aromatic polyesters” (Deepak M. Maher et. al., J. Polym. Sci., Part A: Polym. Chem. 2019, 57, 1516-1526) discloses a cardobisphenol, viz., 2-(2-azidoethyl)-3, 3-bis (4-hydroxyphenyl) isoindolin-1-one (PPH-N₃) synthesized starting from phenolphthalein by a three-step route using simple organic transformations. Aromatic (co) polyesters bearing pendant azido groups were synthesized by low-temperature solution polycondensation of PPH-N₃ or different molar ratios of PPH-N₃ and bisphenol-A (BPA) with aromatic diacid chlorides.

However, bisphenols possessing pendant furyl groups which are connected via alkylene spacer are not reported in the prior art. Therefore, there is a need to develop bisphenols possessing pendant furyl group which is connected via alkylene spacer useful for the synthesis of a range of step growth polymers such as aromatic polycarbonates, polyesters, poly (arylene ether) s, cyanate esters, epoxy resins, etc.

### OBJECTIVES OF THE INVENTION

The main objective of the present invention is to provide bisphenol monomers bearing pendant furyl group of Formula (I).

Another objective of the present invention is to provide (co) polymers of Formula (II) by polycondensation of a mixture of monomers of Formula (I) and bisphenol-A with aromatic diacid chlorides.

Yet another objective of the present invention is to provide a process for the synthesis of (co) polymers of Formula (II) by polycondensation of a mixture of monomers of Formula (I) and bisphenol-A with aromatic diacid chlorides.

### SUMMARY OF THE INVENTION

Accordingly, to accomplish the objectives, the present invention provides phthalimidine-containing bisphenol monomers bearing pendant furyl group of Formula (I) as depicted below:

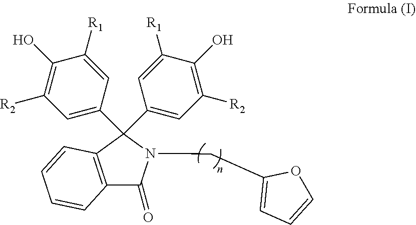

wherein,

R₁ and R₂ are either same or different and selected from the group consisting of Hydrogen, C₁-C₅ substituted or unsubstituted, straight or branched alkyl, and C₁-C₅ substituted or unsubstituted, straight or branched alkoxy;

n=1−8.

In another aspect of the present invention, the substituent of C₁-C₅ alkyl, alkoxy is at least one substituent selected from the group consisting of hydrogen, C₁-C₅ alkyl and C₁-C₅ alkoxy. In an embodiment, the present invention relates to a process for the synthesis of phthalimidine-containing bisphenol monomers compound of Formula (I), wherein the process comprises the step of refluxing phenolphthalein with 1-(furan-2-yl)-alkylamine at a temperature in the range of 140-160° C. for a period in the range of 40-60 hours to obtain the compound of Formula (I).

In another embodiment of the present invention, the process further comprises step of purifying the compound of Formula (I) by recrystallizing using 1:1 (v/v) mixture of ethanol and water.

In another embodiment, the present invention provides a process for the synthesis of phthalimidine-containing bisphenol monomers of Formula (I), wherein the process comprises the steps of:
a) heating the reaction mixture of phenolphthalein (1) and 1-(furan-2-yl)-alkylamine (2) at a temperature in the range of 140-160° C. for a period in the range of 40-60 h;
b) removing the excess 1-(furan-2-yl)-alkylamine (2) from the reaction mixture obtained at step a) under reduced pressure;
c) dissolving the reaction mixture obtained at step b) in a solvent at 25-35° C. and washing the organic layer with water;
d) concentrating the organic layer obtained at step c) under reduced pressure and dissolving the obtained crude product in aqueous sodium hydroxide solution;
e) neutralizing the obtained basic solution with dilute HCl to obtain a solid;
f) filtering the obtained solid at step e) and washing with cold water;
g) recrystallizing the solid product obtained at step f) from 1:1 (v/v) mixture of ethanol and water to obtain the bisphenol monomer of Formula (I).

In another aspect, the solvent in step c) is selected from dichloromethane or ethyl acetate.

Yet another embodiment, the present invention provides (co) polymer of Formula (II) obtained by polycondensation of varying mixtures of monomers of Formula (I) and bisphenol-A with aromatic diacid chlorides. Formula (II) is as depicted below:

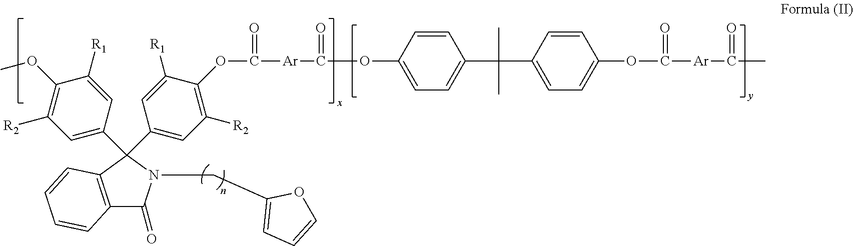

wherein,

R₁ and R₂ are either same or different and selected from the group consisting of H, C₁-C₅ substituted or unsubstituted, straight or branched alkyl, alkoxy;

n=1−8;

x=moles of monomer of Formula (I), y=moles of bisphenol-A; and

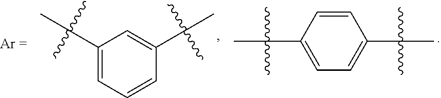 [SMILES: *c1cccc(*)c1 ; *c1ccc(*)cc1]

In yet another aspect, the present invention relates to a process for the synthesis of (co) polymer of Formula (II) using polycondensation of varying mixtures of monomer of Formula (I) and bisphenol-A with aromatic diacid chlorides, wherein the process comprises the steps of:

a. mixing monomer of Formula (I), bisphenol-A and a base under stirring at a temperature in a range of 0-10° C. for 1 hour to obtain a reaction mixture;

b. adding benzyltriethylammonium chloride (BTEAC) and solution of aromatic diacid chloride in a solvent into the reaction mixture obtained at step a) and stirring the reaction mixture vigorously at 2000 rpm at 0-10° C. for 1 h to obtain the compound of Formula (II).

In another embodiment, the present invention provides a process for the synthesis of (co) polymer of Formula (II) obtained by polycondensation of varying mixtures of monomer of Formula (I) and bisphenol-A with aromatic diacid chlorides, wherein the process comprises the steps of:
a. mixing monomer of Formula (I), bisphenol-A (3) and 1M NaOH and stirring the reaction mixture at a temperature in the range of 0-10° C. for 1 h;
b. adding benzyltriethylammonium chloride (BTEAC) and solution of aromatic diacid chloride (4) in a solvent into the reaction mixture obtained at step a);
c. stirring the reaction mixture obtained at step b) vigorously at 2000 rpm at 0-10° C. for 1 h;
d. pouring the reaction mixture obtained at step c) into hot water to precipitate the solid;
e. filtering and washing the solid obtained at step d) with water to remove inorganic impurities;
f. dissolving the solid obtained at step e) in a solvent and pouring the obtained solution into a solvent mixture of methanol and water (1:1, v/v) to obtain the polymer of Formula (II).

In another aspect, the solvent at step b) and step f) is selected from dichloromethane or chloroform.

In yet another aspect, aromatic diacid chloride is selected from isophthaloyl chloride (IPC), terephthaloyl chloride (TPC), mixture of IPC and TPC, 4,4′-biphenyldicarbonyl chloride and 2,6-naphthalenedicarbonyl dichloride.

### BRIEF DESCRIPTION OF THE DRAWINGS

FIG. 1 depicts ¹H-NMR spectrum (in DMSO-d₆) of 2-(furan-2-ylmethyl)-3,3-bis (4-hydroxyphenyl) isoindolin-1-one (BPF).

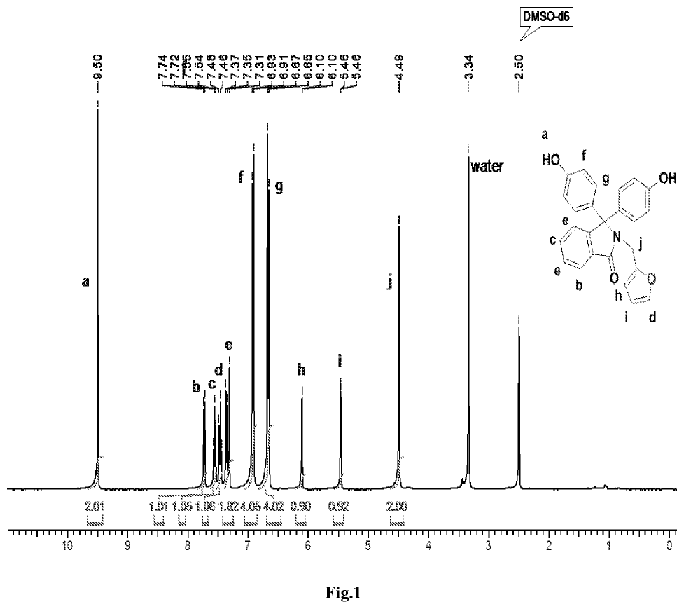

FIG. 2 depicts ¹H-NMR spectrum (in CDCl₃) of aromatic polyester obtained by polycondensation of 2-(furan-2-ylmethyl)-3, 3-bis (4-hydroxyphenyl) isoindolin-1-one with isophthaloyl chloride.

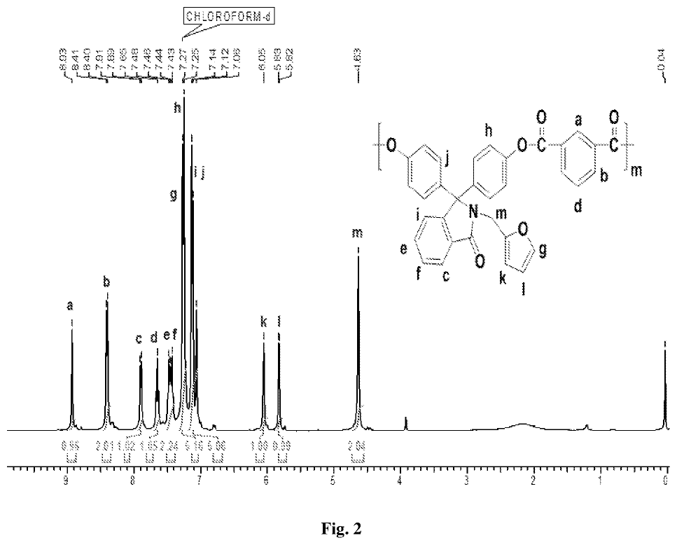

FIG. 3 depicts the preparation of phthalimidine-containing bisphenol monomers of Formula (I).

FIG. 4 depicts the preparation of (co) polymer of Formula (II).

### DETAILED DESCRIPTION OF THE INVENTION

Accordingly, to accomplish the objectives, the present invention provides phthalimidine-containing bisphenol monomers bearing pendant furyl group of Formula (I) as depicted below:

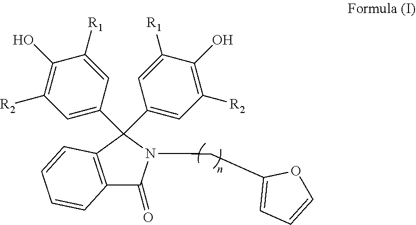

wherein,

R₁ and R₂ are either same or different and selected from the group consisting of H, C₁-C₅ substituted or unsubstituted, straight or branched alkyl, alkoxy;

n=1−8.

In another embodiment, the present invention provides a process for the synthesis of phthalimidine-containing bisphenol monomers of Formula (I), wherein the process comprises the steps of:
a) heating the reaction mixture of phenolphthalein (1) and 1-(furan-2-yl)-alkylamine (2) at a temperature in the range of 140-160° C. for a period in the range of 40-60 h;
b) removing the excess 1-(furan-2-yl)-alkylamine (2) from the reaction mixture obtained at step a) under reduced pressure;
c) dissolving the reaction mixture obtained at step b) in a solvent at 25-35° C. and washing the organic layer with water;
d) concentrating the organic layer obtained at step c) under reduced pressure and dissolving the obtained crude product in aqueous sodium hydroxide solution;
e) neutralizing the obtained basic solution with dilute HCl to obtain a solid;
f) filtering the obtained solid at step e) and washing with cold water;
g) recrystallizing the solid product obtained at step f) from 1:1 (v/v) mixture of ethanol and water to obtain the bisphenol monomer of Formula (I).

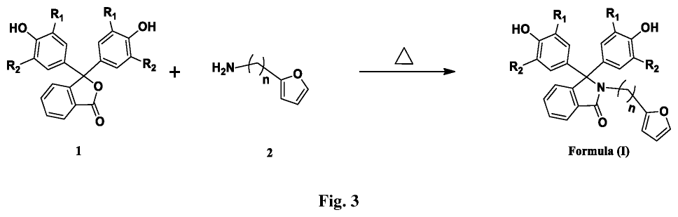

The synthesis of bisphenol monomers of Formula (I) is depicted below in FIG. 3.

The solvent at step c) is selected from dichloromethane or ethyl acetate.

Yet another embodiment of the present invention provides (co) polymer of Formula (II) obtained by polycondensation of varying mixtures of monomers of Formula (I) and bisphenol-A with aromatic diacid chlorides. Formula (II) is as depicted below:

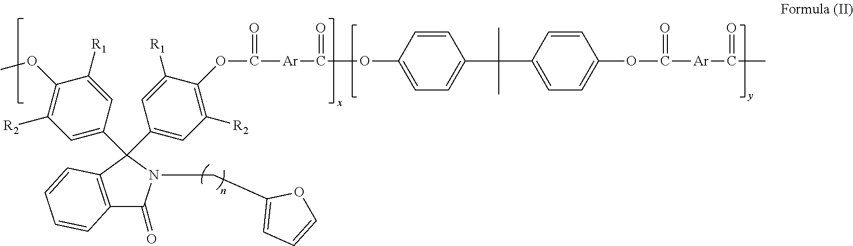

wherein,

R₁ and R₂ are either same or different and selected from the group consisting of H, C₁-C₅ substituted or unsubstituted, straight or branched alkyl, alkoxy;

n=1−8;

x=moles of monomer of Formula (I), y=moles of bisphenol-A; and

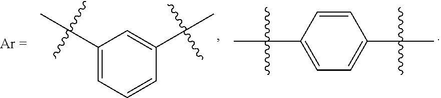 [SMILES: *c1cccc(*)c1 ; *c1ccc(*)cc1]

In yet another aspect, the present invention relates to a process for the synthesis of (co) polymer of Formula (II) using polycondensation of varying mixtures of monomer of Formula (I) and bisphenol-A with aromatic diacid chlorides, wherein the process comprises the steps of:
a. mixing monomer of Formula (I), bisphenol-A and a base under stirring at a temperature in a range of 0-10° C. for 1 hour to obtain a reaction mixture; and
b. benzyltriethylammonium chloride (BTEAC) and solution of aromatic diacid chloride in a solvent into the reaction mixture obtained at step a) and stirring the reaction mixture vigorously at 2000 rpm at 0-10° C. for 1 h to obtain the compound of Formula (II).

In another embodiment of the present invention, the base is selected from sodium hydroxide, potassium hydroxide, cesium hydroxide and so on.

In another embodiment of the present invention, the solvent is dichloromethane or chloroform.

In another embodiment of the present invention, the process further comprises step of purifying the compound of Formula (II) by recrystallizing using 1:1 (v/v) mixture of ethanol and water

Another embodiment of the present invention provides a process for the synthesis of (co) polymer of Formula (II) obtained by polycondensation of varying mixtures of monomer of Formula (I) and bisphenol-A with aromatic diacid chlorides, wherein the process comprises the steps of:

a) mixing monomer of Formula (I), bisphenol-A (3) and 1M NaOH and stirring the reaction mixture at a temperature in the range of 0-10° C. for 1 h;
b) adding benzyltriethylammonium chloride (BTEAC) and solution of aromatic diacid chloride (4) in a solvent into the reaction mixture obtained at step a);
c) stirring the reaction mixture obtained at step b) vigorously at 2000 rpm at 8-10° C. for 1 h;
d) pouring the reaction mixture obtained at step c) into hot water to precipitate the solid;
e) filtering and washing the solid obtained at step d) with water to remove inorganic impurities;
f) dissolving the solid obtained at step e) in a solvent and pouring the obtained solution into a solvent mixture of methanol and water (1:1, v/v) to obtain the polymer of Formula (II).

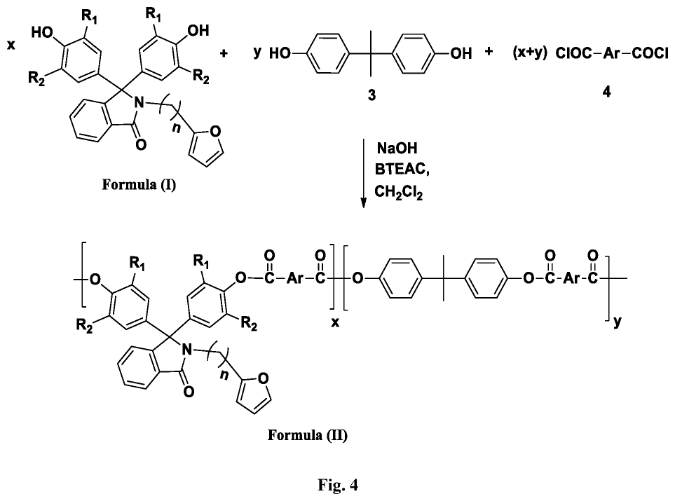

The preparation of polymer of Formula (II) is depicted in FIG. 4

The solvent at step b) and step f) is selected from dichloromethane or chloroform. Aromatic diacid chloride is selected from isophthaloyl chloride (IPC), terephthaloyl chloride (TPC), mixture of IPC and TPC, 4,4′-biphenyldicarbonyl chloride and 2,6-naphthalenedicarbonyl dichloride.

In an embodiment, the present invention relates to a polymer comprising the recurring units of compound of Formula (I).

In another embodiment of the present invention, the polymers are selected from aromatic polycarbonates, polyether sulfones, and polyetherketones.

In another embodiment of the present invention, the incorporation of phthalimidine moieties offers advantages such as improved thermal characteristics. Glass transition temperature (T_{g}) values of (co) polyesters were in the range 202-242° C. and temperature for 10% weight loss (T₁₀) in thermogravimetric analysis under nitrogen atmosphere were in the range 415-452° C. (Co) polyesters demonstrated improved solubility characteristics and were soluble in common organic solvents such as dichloromethane, chloroform and tetrahydrofuran. The solution processability of polymers to form films is advantageous in the formation of membrane materials for gas separation and in coatings.

In another embodiment of the present invention, the polymers possessing pendant furyl groups based on phthalimidine containing cardobisphenols provide interesting opportunities for post-polymerization modification and cross-linking reactions with the use of appropriately functionalized maleimide and bis-maleimide, respectively.

In another embodiment of the present invention, the thermo-reversible cross-linking via bis-maleimide allows the polymers to improve thermal and mechanical properties with an added advantage of recyclability unlike conventional thermosetting resins.

### EXAMPLES

Following examples are given by way of illustration and therefore should not be construed to limit the scope of the invention.

### Example—1: Synthesis of 2-(furan-2-ylmethyl)-3, 3-bis (4-hydroxyphenyl) isoindolin-1-one

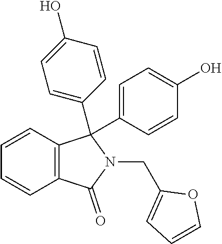 [SMILES: O=C1c2ccccc2C(c2ccc(O)cc2)(c2ccc(O)cc2)N1Cc1ccco1]

Into a 250 mL two-necked round bottom flask equipped with a reflux condenser, an addition funnel and a magnetic stirring bar were charged phenolphthalein (1) (10.0 g, 31.41×10⁻³ mol), and 1-(furan-2-yl)-alkyl-amine (2) (36.61 g, 376.9×10⁻³ mol). The reaction mixture was heated at 150° C. for 48 h. The excess 1-(furan-2-yl) methanamine was removed under reduced pressure. The reaction mixture was cooled to 30° C., dissolved in ethyl acetate (200 mL) and washed with water (2×100 mL). The ethyl acetate layer was separated, dried over anhydrous sodium sulfate and filtered. Ethyl acetate was removed under reduced pressure and the obtained product was dissolved in aqueous sodium hydroxide solution, followed by neutralization with dilute hydrochloric acid. The obtained precipitate was separated out by filtration and washed with cold water. The product was purified by recrystallization from a mixture of ethanol and water (1:1, v/v). Yield: 4.9 g (35%). Melting Point: 284° C.

FT-IR (KBr, cm¹): 3385 (OH), 1667 (C═O), 1507 (C═C)

¹H NMR (DMSO-d₆, δ ppm): 9.50 (br. s, 2H), 7.73 (d, 1H), 7.55 (t, 1H), 7.47 (d, 1H), 7.37-7.31 (m, 2H), 6.92 (d, 4H), 6.66 (d, 4H), 6.66 (d, 1H), 5.46 (d, 1H), 4.49 (s, 2H).

¹³C NMR (DMSO-d₆, δ ppm): 167.4, 157.2, 152.0, 150.9, 141.5, 132.7, 130.6, 129.8, 129.4, 128.3, 124.0, 123.2, 115.4, 110.5, 107.1, 74.9, 74.9, 39.3.

### Example—2

### Representative Procedure for Synthesis of Aromatic (Co) Polyesters Bearing Pendant Furyl Groups

Into an oven-dried 100 mL two-necked round bottom flask equipped with a high-speed mechanical stirrer and an addition funnel were placed BPF (0.750 g, 1.88×10⁻³), BPA (0.430 g, 1.88×10⁻³) and 1M NaOH (8.5 mL). The reaction mixture was stirred at 10° C. for 1 h. Afterwards, benzyltriethylammonium chloride (BTEAC) (57 mg) was added into the reaction mixture. The solution of isophthaloyl chloride (IPC) (0.7669 g, 3.776×10⁻³ mol) dissolved in dichloromethane (7 mL) was added in one lot into the reaction mixture and the mixture was stirred vigorously at 2000 rpm at 10° C. for 1 h. The reaction mixture was poured into hot water; the precipitated polymer was filtered, and washed several times with water to remove the inorganic impurities. Polyester was dissolved in dichloromethane (25 mL) and the solution was poured into a mixture of methanol and water (1:1, v/v) (1000 mL) to precipitate the polymer. The polyester was filtered, washed with methanol and dried at 50° C. under reduced pressure for 24 h.

Copolyesters given in the table-1 below were also synthesized by polycondensation of varying molar ratios of BPF and bisphenol-A with aromatic diacid chlorides such as terephthaloyl chloride or isophthaloyl chloride using the similar procedure.

TABLE 1
% Molar composition of copolyestersbearing pendant furyl groups calculated by ¹H NMR spectra

| | | Feed | Observed |
|---|---|---|---|
| Polyester | | BPF, | BPF, |
| code | Copolyester | mol % | mol % |
| FPE-2 | 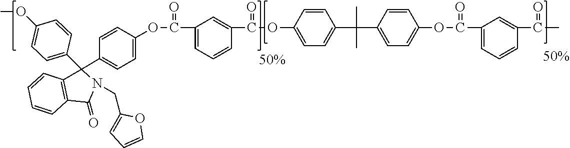 | 50 | 50 |
| FPE-3 | 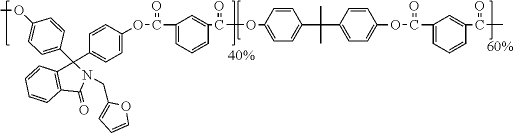 | 40 | 49.5 |
| FPE-4 | 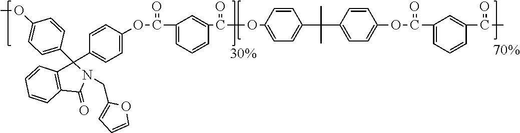 | 30 | 29.5 |
| FPE-5 | 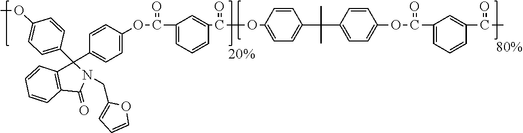 | 20 | 20 |
| FPE-6 | 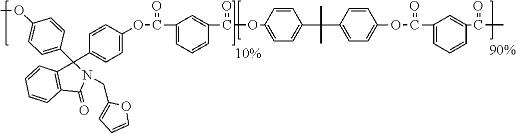 | 10 | 10 |

Inherent viscosity values of (co) polyesters were found in the range 0.51-1.1 dL/g and GPC data showed Mn values in the range 25,400-48,900 g/mol. These data given in table 2 below specified the formation of reasonably high molecular weight (co) polyesters. As expected from a step growth polymerization mechanism, the dispersity values were close to 2.

TABLE 2
Results of synthesis of (co)polyesters bearing pendant furyl groups

| | | | | | Molecular weight | | |
|---|---|---|---|---|---|---|---|
| Polyester | BPF | BPA | IPC | ηᵢₙₕ | (g mol⁻¹) ^{b} | | Dispersity |
| code | mol % | mol % | mol % | dL/g^{a} | Mn | Mw | Mw/Mn |
| FPE-1 | 100 | 00 | 100 | 0.81 | 31,700 | 68,700 | 2.1 |
| FPE-2 | 50 | 50 | 100 | 0.51 | 25,400 | 52,400 | 2.0 |
| FPE-3 | 40 | 60 | 100 | 0.89 | 37,600 | 79,400 | 2.1 |
| FPE-4 | 30 | 70 | 100 | 1.00 | 42,500 | 80,000 | 1.9 |
| FPE-5 | 20 | 80 | 100 | 1.10 | 48,900 | 94,400 | 1.9 |
| FPE-6 | 10 | 90 | 100 | 0.74 | 27,100 | 53,100 | 1.9 |

### Example—3: Thermal Properties of (co) Polyesters Bearing Pendant Furyl Groups

Thermal behavior of (co) polyesters bearing pendant furyl groups was evaluated by thermogravimetric (TG) analysis at a heating rate of 10° C./min under nitrogen atmosphere. The Tg values of (co) polyesters were evaluated by DSC at a heating rate of 10° C./min under nitrogen atmosphere. The data obtained were given in the table below.

TABLE 3
Thermal properties of (co) polyesters bearing pendant furyl groups

| | | | | Char |
|---|---|---|---|---|
| (Co)polyester | | Tg | T₁₀ | Yield |
| Code | (Co)polyester | (° C.)^{a} | (° C.)^{b} | (%)^{c} |
| FPE-1 | 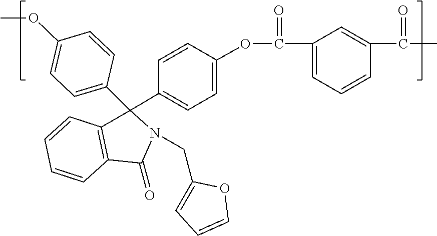 | 242 | 415 | 43 |
| FPE-2 | 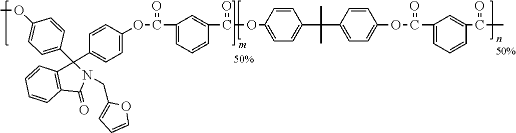 | 223 | 417 | 37 |
| FPE-3 | 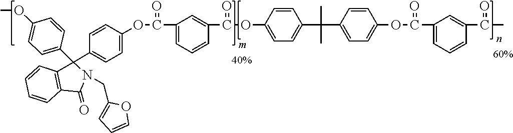 | 216 | 425 | 35 |
| FPE-4 | 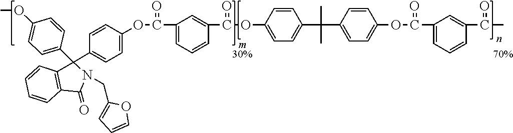 | 209 | 437 | 33 |
| FPE-5 | 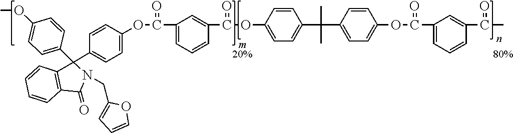 | 203 | 446 | 27 |
| FPE-6 | 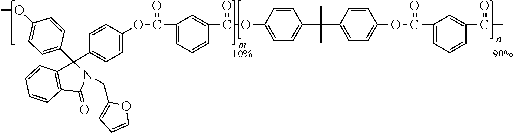 | 202 | 452 | 23 |

^{a}Glass transition temperature
^{b}10% Weight loss on TG curves at a heating rate of 10º C./min under nitrogen atmosphere
^{c}% Char yield measured at 800° C.

The single value of Tg of all the copolyesters support the formation of random copolymers was inferred from ¹³C NMR spectroscopy studies. The homopolyester FPE-1 containing cardophthalimidine moieties exhibited Tg value of 242° C. It is to be noted that reported Tg value of polyester obtained by polycondensation of BPA with IPC is 180° C. As anticipated, Tg values decreased with increase in mol % incorporation of BPA in the series of (co) polyesters. The T₁₀ values of (co) polyesters were in the range 415-452° C. which indicated their higher thermal stability.

### ADVANTAGES OF THE INVENTION

A simple and convenient process for the synthesis of phthalimidine-containing bisphenol monomers bearing pendant furyl group linked via an alkylene spacer is developed
Readily available and inexpensive chemicals are involved in the process
These phthalimidine-containing cardobisphenols are potentially useful for the synthesis of a range of step growth polymers such as aromatic polycarbonates, polyesters, poly (arylene ether)s, cyanate esters, epoxy resins, etc.
The polymers exhibit desirable combination of properties such as improved thermal properties accompanied by solubility in common organic solvents. These properties offer advantages in terms of solution processability and applications as high temperature thermoplastics.

1-10. (canceled)

11. A phthalimidine-containing bisphenol monomer compound of Formula (I): 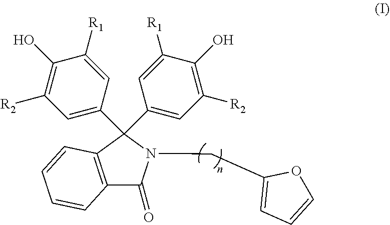
where:
R₁ and R₂ are independently selected from the group consisting of —H, substituted or unsubstituted straight or branched C₁-C₅ alkyl, and substituted or unsubstituted straight or branched C₁-C₅ alkoxy; and
n is an integer from 1 to 8.

12. The phthalimidine-containing bisphenol monomer compound of claim 11, wherein R₁ and R₂ are independently selected from the group consisting of:
—H;
unsubstituted straight or branched C₁-C₅ alkyl;
straight or branched C₁-C₅ alkyl substituted with a straight or branched substituted or unsubstituted C₁-C₅ alkyl or a straight or branched substituted or unsubstituted C₁-C₅ alkoxy;
unsubstituted straight or branched C₁-C₅ alkoxy; and
straight or branched C₁-C₅ alkoxy substituted with a straight or branched substituted or unsubstituted C₁-C₅ alkyl or a straight or branched substituted or unsubstituted C₁-C₅ alkoxy.

13. A process for synthesizing the phthalimidine-containing bisphenol monomer compound of claim 11, the process comprising:
refluxing phenolphthalein with 1-(furan-2-yl)-alkylamine at 140° C. to 160° C. for 40 hours to 60 hours to obtain the compound of Formula (I).

14. The process according to claim 13, further comprising:
purifying the compound of Formula (I) by recrystallizing using 1:1 (v/v) mixture of ethanol and water.

15. A (co) polymer comprising monomers of the phthalimidine-containing bisphenol monomer compound according to claim 1, the (co) polymer having of Formula (II): 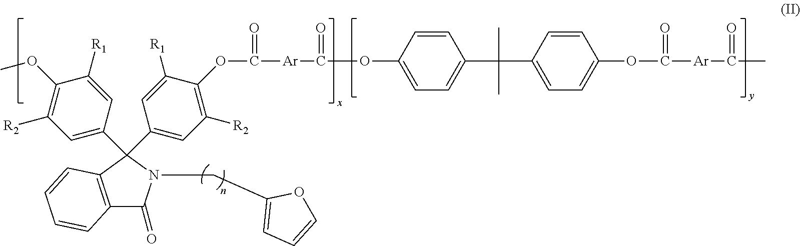
wherein,
R₁ and R₂ are independently selected from the group consisting of —H, substituted or unsubstituted straight or branched C₁-C₅ alkyl, and substituted or unsubstituted straight or branched C₁-C₅ alkoxy;
n is an integer from 1 to 8;
x is a number of moles of the phthalimidine-containing bisphenol monomer compound;
y is a number of moles of bisphenol-A; and
Ar is chosen from

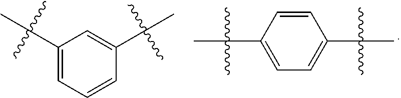 [SMILES: *c1cccc(*)c1 ; *c1ccc(*)cc1]

16. The (co) polymer of claim 15, wherein R₁ and R₂ are independently selected from the group consisting of:
—H;
unsubstituted straight or branched C₁-C₅ alkyl;
straight or branched C₁-C₅ alkyl substituted with a straight or branched substituted or unsubstituted C₁-C₅ alkyl or a straight or branched substituted or unsubstituted C₁-C₅ alkoxy; unsubstituted straight or branched C₁-C₅ alkoxy; and
unsubstituted straight or branched C₁-C₅ alkoxy; and
straight or branched C₁-C₅ alkoxy substituted with a straight or branched substituted or unsubstituted C₁-C₅ alkyl or a straight or branched substituted or unsubstituted C₁-C₅ alkoxy.

17. A process for synthesizing the (co) polymer of claim 15, the process comprising:
(a) mixing the phthalimidine-containing bisphenol monomer compound, bisphenol-A, and a base under stirring at from 0° C. to 10° C. for 1 hour to obtain a reaction mixture;
(b) adding, into the reaction mixture of (a), benzyltriethylammonium chloride and a solution of aromatic diacid chloride in a solvent and stirring the reaction mixture vigorously at 2000 rpm at 0° C. to 10° C. for 1 hour to obtain the compound of Formula (II).

18. The process according to claim 17, wherein:
the base is chosen from sodium hydroxide, potassium hydroxide, or cesium hydroxide; and
the solvent is chosen from dichloromethane or chloroform.

19. The process according to claim 17, wherein the aromatic diacid chloride is selected from isophthaloyl chloride, terephthaloyl chloride, 4,4′-biphenyldicarbonyl chloride, 2,6-naphthalenedicarbonyl dichloride, or mixtures thereof.

20. A polymer comprising recurring units of the phthalimidine-containing bisphenol monomer compound according to claim 11, wherein the polymer is selected from aromatic polycarbonates, polyether sulfones, or polyetherketones.
